(19) United States
(12) Patent Application Publication
(10) Pub. No.: US 2005/0075556 A1
(43) Pub. Date: Apr. 7, 2005

(54) Method for remotely controlling medical apparatuses and device therefor

(76) Inventor: William Pan, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/676,103

(22) Filed: Oct. 2, 2003

(51) Int. Cl. A61B 5/05; A61B 8/00; A61B 6/00

(52) U.S. Cl. 600/407; 600/437; 600/476; 600/118; 378/4

(57) ABSTRACT

A method for remotely controlling medical apparatuses and the method thereof are proposed. The medical apparatuses are used to take internal images of the human body, and have control command sets that comply with the DICOM specifications. In this method, a server is first built and connected to the medical apparatuses. A remote device is then built and connected to the server. Next, the remote device executes a remote control program to generate at least a DICOM control command, which is then sent to the server. The server then executes a communication program to perform control communications with the medical apparatuses. The medical apparatuses can thus be controlled to take images of the inside of human body for generating of digital image data complying with the DICOM specifications. The object of remote control for integrating medical apparatuses can thus be accomplished.

### BACKGROUND OF THE INVENTION

1. Field of the Invention

The present invention relates to a method for controlling medical apparatuses and a device therefor. More particularly, the present invention relates to a method and device therefor that makes the use of wired or wireless network architecture to remotely control a server for picking images generated by at least one medical imaging apparatus.

2. Background of the Invention

As computer technology continuously improves, the cooperation of computer technologies and medical instruments permit more advanced medical techniques to be practiced in the medical field. For instance, 3D computer imaging techniques can be exploited in supersonic medical apparatuses to show internal images of the human body or the face and shape of a fetus. Other medical imaging apparatuses such as a CAT scanner and an endoscope can digitize their produced image by using computer techniques and then store or further manipulate them. Through combination of images, the cause of sickness within the human body can be found much more rapidly and hence, greatly uplifting the success rate of surgical operations.

Medical imaging apparatuses such as an angiograph, an ultrasound, an endoscope, an intraoral camera or a CAT scanner are used to take internal images of the human body and generate medical image signals. Image data generated by these different kinds of medical apparatuses are enormous and do not have the same image data properties. Further the image data of medical apparatuses produced by different manufacturers are incompatible and therefore, it is difficult to widely apply or share the resources.

Therefore, the medical world has formulated a Digital Imaging Communications in Medicine (DICOM) specifications. All manufacturers can manufacture medical apparatuses according to the DICOM specifications to achieve widespread application and resources sharing of generated image data. Different apparatuses can therefore communicate or control mutually through the DICOM specifications. Hence, a computerized medical environment without national boundaries and obstacles can be constructed

In the prior art, even though new-type medical apparatuses conform to the DICOM specifications, they are limited to have merely the same data format, and have no control system for integrated applications. In present, most hospitals are still using many old-types of medical apparatuses, which do not comply to the DICOM specifications.

### SUMMARY OF THE INVENTION

The primary object of the present invention is to provide a method for remotely controlling a medical apparatus. The said medical apparatus is used to take internal images of human body and have control command sets complying with the DICOM specifications hence, the generated digital image data will comply with the DICOM specifications. In the said method, firstly, a server is built and connected to the medical apparatus and then a remote device is constructed to join with the said server. Next, the remote device executes a remote control program to generate at least one DICOM control command, which is sent to the medical apparatus via the server. The server then executes a communication program to perform control communications with the medical apparatus. The medical apparatus is commanded to take images within human body for generating digital image data and store them in the server or sent them to the remote device. Thus, this integrated medical apparatus is complied with the DICOM specifications and can be remotely controlled.

The second object of the present invention is to provide a method for remotely controlling a medical apparatus, which is used to take internal images of human body for generating medical image signals. In this method, a digital conversion device is firstly built and connected to the medical apparatus. The digital conversion device is used to convert the medical image signals into digital image data to comply with the DICOM specifications. Secondly, a server is constructed and connected to the said digital conversion device. Next, a remote device is placed and connected to the server. That is, the remote device is connected to the digital conversion device via the server. The remote device then executes a remote control program to generate at least one DICOM control command that is sent to the server. The server then executes a communication program to perform control communications with the digital conversion device so as to control the digital conversion device to capture or store the digital image data into the server or send the digital image data to the remote device. Thereby, conventional medical apparatuses that do not comply with the DICOM specifications can thus be integrated and remotely controlled.

Another object of the present invention is to provide a device for remotely controlling a medical apparatus. The device comprises at least a medical apparatus, a digital conversion device, a server, an image storage database and at least a remote device. The medical apparatus is used to take inside image of human body for generating medical image signals. The digital conversion device is connected to the medical apparatus and used to convert the digital image signals into a set of digital image data that comply with the DICOM specifications. The server is connected to the medical apparatus or the digital conversion device and is capable of executing at least one communication program to perform control communication functions with the medical apparatus or the digital conversion device. The image storage database is connected to the server and used to store the digital image data. The remote device is connected to the server and is capable of generating at least one DICOM control command that is sent to the medical apparatus via the server. Thereby, all medical apparatuses can be integrated to form a computerized medical environment without national boundaries and obstacles.

Yet another object of the present invention is to provide with the above-mentioned device to remotely control a medical apparatus, in which the digital conversion device, the server and the remote device are all equipped with network interface units, hence, that they can be interconnected using a network. The network interface units can utilize a wired or wireless network to achieve network connection, hence allowing remote control and data transmission.

### BRIEF DESCRIPTION OF THE DRAWINGS

The various objects and advantages of the present invention will be more readily understood from the following detailed description when read in conjunction with the appended drawing, in which:

FIG. 1 is an architecture diagram of a system of the present invention for remotely controlling medical apparatuses;

FIG. 2 is a hardware architecture diagram of the digital conversion device of the present invention;

FIG. 3 is an internal block diagram of the server of the present invention;

FIG. 4 is an internal block diagram of the remote device of the present invention;

FIG. 5 is a flowchart of the method of the present invention for remotely controlling a medical apparatus; and

FIG. 6 is a flowchart of the method of the present invention for remotely controlling the digital conversion device.

### DETAILED DESCRIPTION OF THE EMBODIMENTS

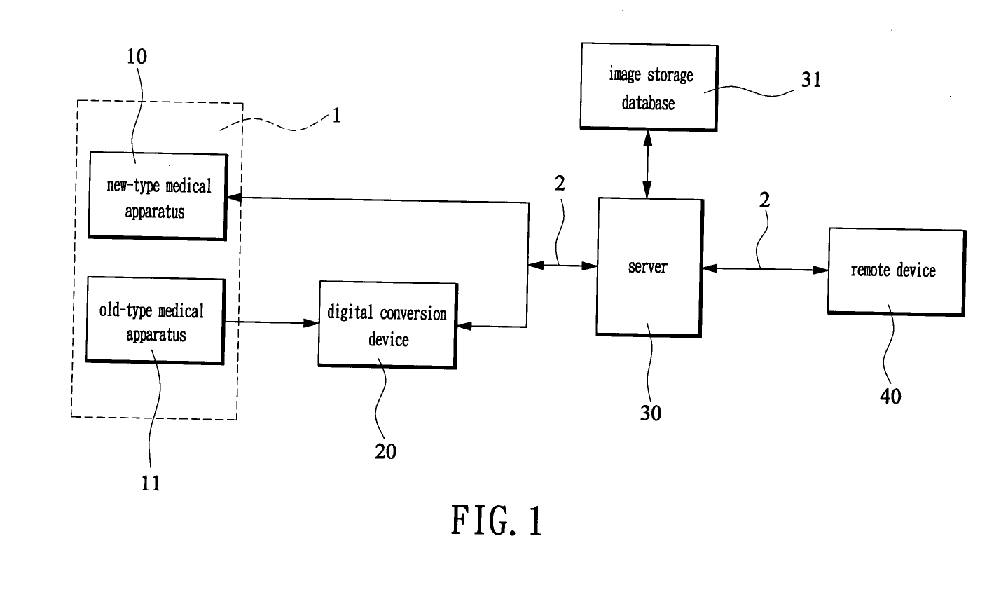

As shown in FIG. 1, medical apparatuses 1 of the present invention include any apparatus capable of taking internal images of human body to generate medical image signals. These medical apparatuses include new-type medical apparatuses 10, which comply with the DICOM specifications and old-type medical apparatuses 11, which do not comply with the DICOM specifications.

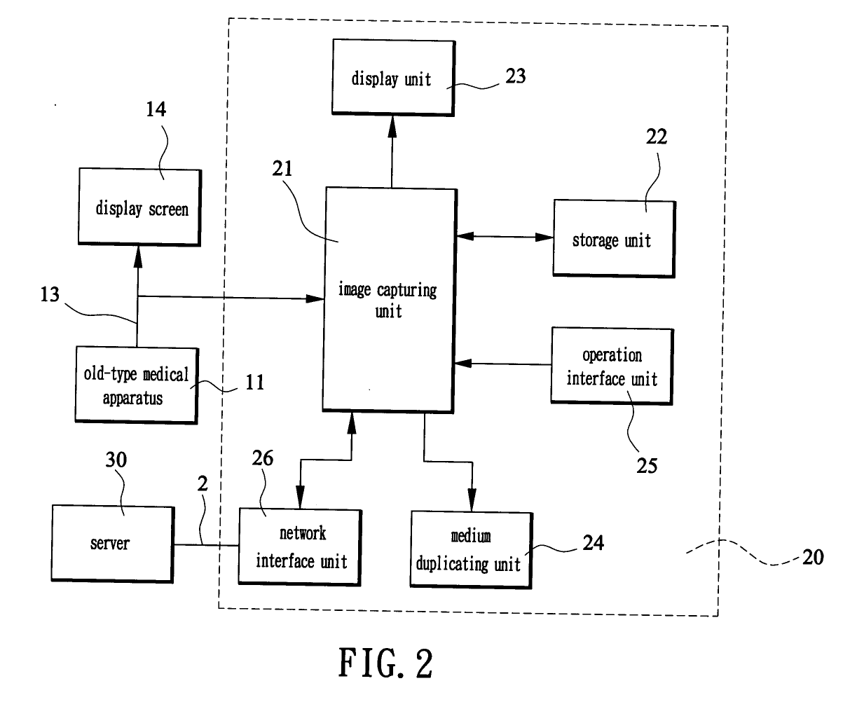

In order to integrate medical image signals generated by the old-type medical apparatus, in the present invention, a digital conversion device 20 is connected to the old-type medical apparatus 11. The main function of the digital conversion device 20 is to convert the medical image signals into digital image data complying with the DICOM specifications. As shown in FIG. 2, the digital conversion device 20 comprises an image capturing unit 21, a storage unit 22, a display unit 23, a medium duplicating unit 24, a human/machine interface unit 25 and a network interface unit 26.

Wherein, the image capturing unit 21 is connected to the old-type medical apparatus 11 and used to capture the medical image signals and convert them into digital image data complying with the DICOM specifications. Generally, an image signal line 13 is provided to the old-type medical apparatus 11 and connected to a display screen 14 for displaying the captured image. In the present invention, the image capturing unit 21 is connected to the image signal line 13 to capture the image signal. The image capturing unit 21 utilizes a computer image-capturing card to extract the image signal and convert these into digital image files of AVI or MPEG format. However, it is still necessary to modify the encoded format of the image-capturing card and convert the image files into digital image data that comply with the DICOM specifications.

The storage unit 22 can be a hard disk drive, which is connected to the image capturing unit 21 for storing the generated digital image data. The display unit 23 can be a screen display, and is used to display the digital image data captured by the image capturing unit 21 or the digital image data stored in the storage unit 22. The medium duplicating unit 24 can be a CD-RW recorder, and is used to duplicate the digital image data stored in the storage unit 22 into a medium format such as VCD for long-term preservation or education purposes. The operation interface unit 25 includes devices such as keyboard, mouse or touch pad, and is used to control actions such as storage, deletion, search, medium duplication or transmission of the digital image data in the storage unit 22.

The network interface unit 26 is connected to the image capturing unit 21, and is joined to a network 2 for sending the digital image data to other computers or apparatuses conforming to the DICOM specifications. The network 2, which connects to the network interface unit 26, can be a wired or a wireless network. Therefore, the network interface unit 26 can be a wired network interface such as a TCP/IP network card for wired network transmission or a wireless network interface such as an 802.11 wireless network card for wireless network transmission.

Through the function of the digital conversion device 20, the old-type medical apparatus 11, like the new-type medical apparatus 10, is able to generate digital image data that comply with the DICOM specifications. The only discrepancy between them is that the new-type medical apparatus 10 has control command sets comply with the DICOM specifications, and can be directly controlled by DICOM control commands to act. The old-type medical apparatus 11 cannot directly accept the DICOM control command sets. It is necessary to generate digital image data and store them in the digital conversion device and then controlled by the DICOM control command sets.

Reference is again made to FIG. 1. The present invention also comprises a server 30,which is connected to the new-type medical apparatus 10 or the digital conversion device 20. The server 30 is also connected to an image storage database 31 for storing enormous amount of digital image data. Several remote devices 40 can be connected to the server 30 via the network 2. The remote device 40 can remotely generate DICOM control commands to control the medical apparatuses 1 or the digital conversion device 20 via the server for purpose of generating, capturing, storage or transmission of the digital image data.

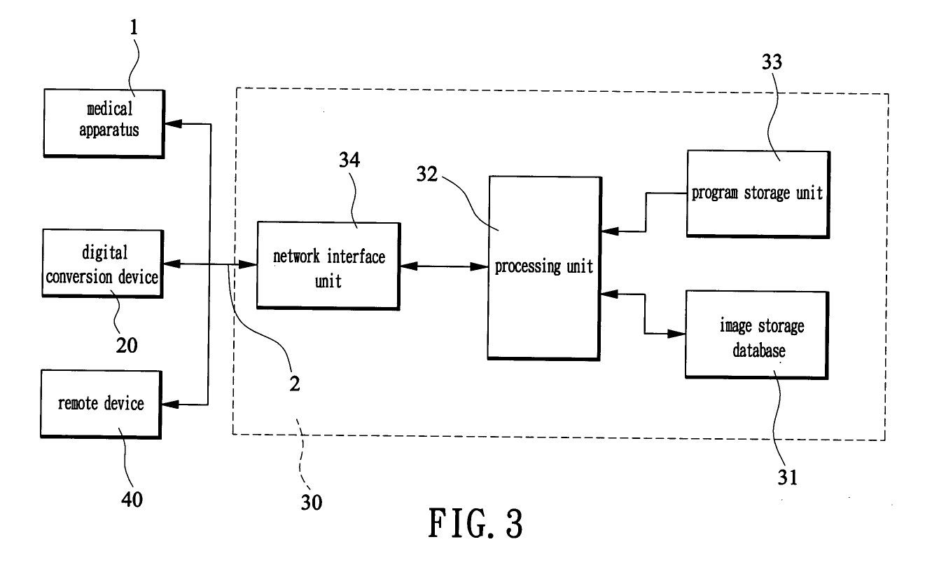

As shown in FIG. 3, wherein the server 30 comprises a processing unit 32, a program storage unit 33 and a network interface unit 34. The program storage unit 33 stores at least a communication program therein. The communication program is used to communicate with the medical apparatuses or the digital conversion device for controlling generation, selection, storage or transmission of the digital image data. The processing unit 32 is connected to the program storage unit 33 and used to execute the communication program to perform control and communication with the medical apparatuses. The network interface unit 34 is connected to the processing unit 32, and can connect to the remote device 40 via a network 2 for sending the digital image data to the remote device, and can also connect the medical apparatuses 1 or the digital conversion device 20 for receiving the generated digital image data. The network interface unit 34 can be a wired or wireless network interface like that of the digital conversion device 20.

The remote device 40 can remotely control the medical apparatuses 1 or the digital conversion device 20 via the server 30. The remote device 40 can be a desktop computer, a notebook computer or a personal digital assistant (PDA). The remote device 40 generates at least a DICOM control command sent to the server 30. Through the communication program of the server 30 with the medical apparatuses 1 or the digital conversion device 20, the medical apparatuses 1 or the digital conversion device 20 is controlled to perform generation, capturing, storage or transmission of the digital image data.

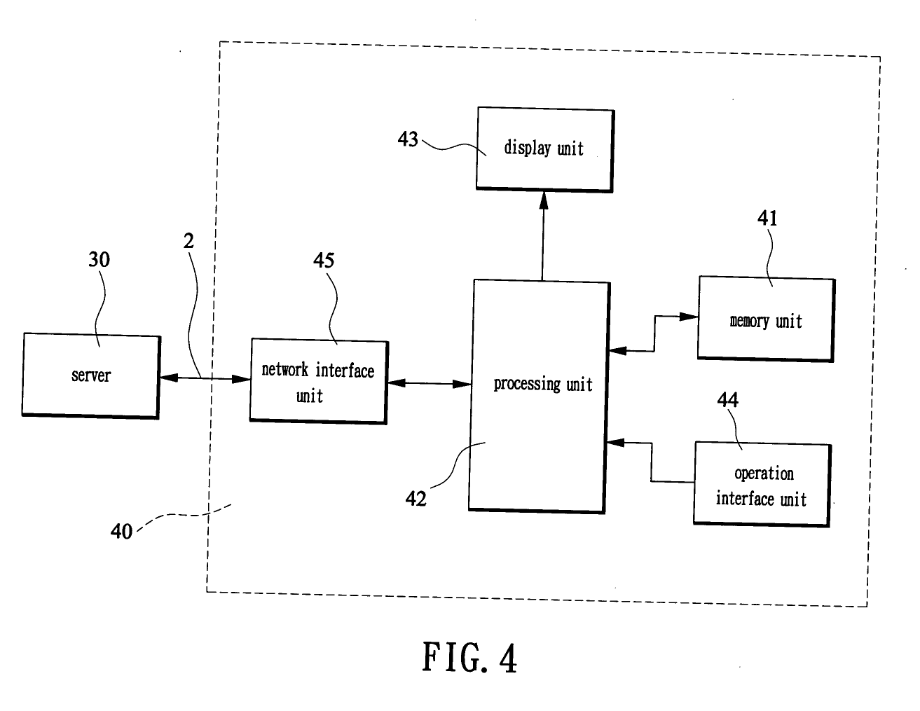

As shown in FIG. 4, the remote device 40 at least comprises a memory unit 41, a processing unit 42, a display unit 43, an operation interface unit 44 and a network interface unit 45. The memory unit 41 is used to store at least a remote control program. The processing unit 42 is connected to the memory unit 41, and is used to execute the remote control program for generating at least a DICOM control command. The display unit 43 is connected to the processing unit 42, and is used to display execution results of the remote control program or the digital image data. The operation interface unit 44 is used to enable a user to control the remote device 40 and execution of the remote control program. The network interface unit 45 is connected to the processing unit 42, and can connect to the server 30 via a network 2 to send the DICOM control commands to the server 30 or receive the digital image data from the server 30. Similarly, the network interface unit 45 can be a wired or wireless network interface device.

Because the image data format and communication format have been defined in the DICOM specifications, the present invention designs a complete control system, whose internal transmission and communication data formats fully comply with the DICOM specifications.

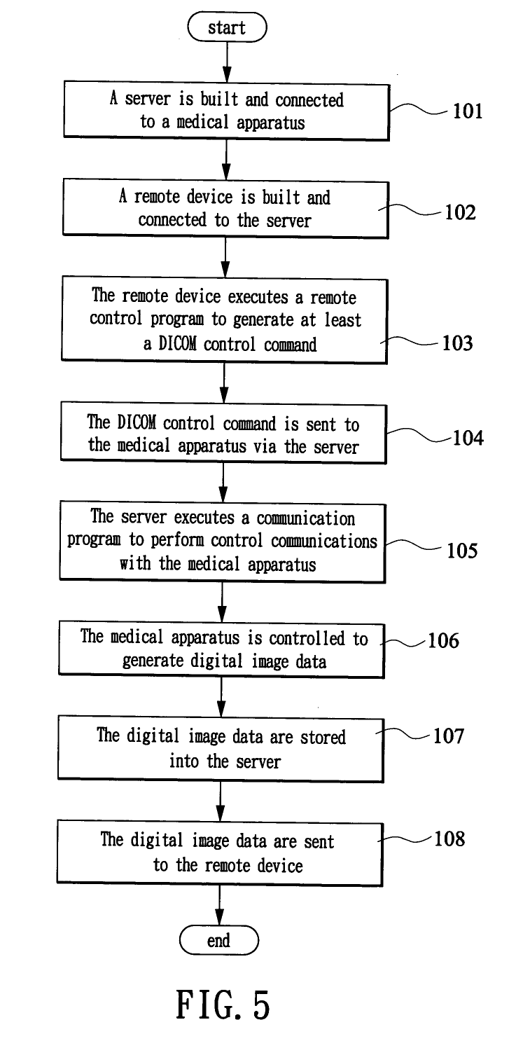

FIG. 5 is a flowchart of the method of the present invention for remotely controlling a medical apparatus. First, a server 30 is built and connected to the medical apparatuses 1 (Step 101). The server 30 uses the control command sets that comply with the DICOM specifications to connect the medical apparatus 1. Next, a remote device 40 is placed and connected to the server 30 (Step 102). The remote device 40 executes a remote control program to generate at least one DICOM control command (Step 103). The DICOM control command is sent to the medical apparatus 1 via the server 30 (Step 104). The server 30 then executes a communication program to perform control communication with the medical apparatus 1 (Step 105). The medical apparatus 1 is controlled to take internal images of the human body for generating digital image data (Step 106)). Finally, the digital image data are stored in the server 30 (Step 107) or sent to the remote device 40.

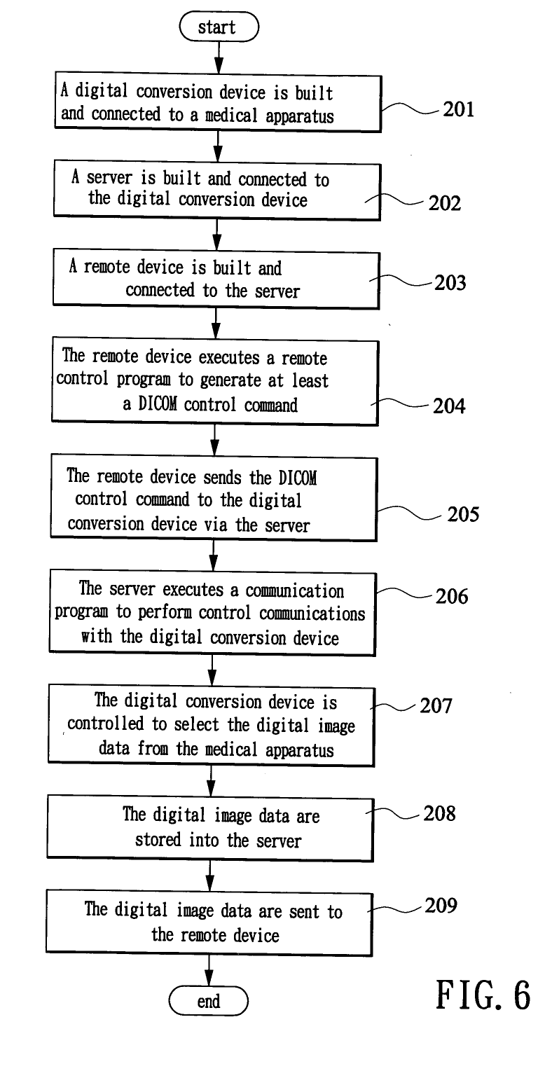

FIG. 6 is a flowchart of the method of the present invention for remotely controlling the digital conversion device. First, a digital conversion device 20 is built and connected to a medical apparatus 1 (Step 201). The digital conversion device 20 is used to convert the medical image signals into digital image data conforming to the DICOM specifications. A server 30 is then placed and connected to the digital conversion device 20 (Step 202). A remote device 40 is built and connected to the server 30, and is also connected to the digital conversion device 20 via the server 30 (Step 203). The remote device 40 executes a remote control program to generate at least a DICOM control command (Step 204). The remote device 40 then sends the DICOM control command to the digital conversion device 20 via the server 30 (Step 205). The server 30 executes a communication program to perform control communication with the digital conversion device 20 (Step 206). The digital conversion device 20 is commanded to capture the digital image data from the medical apparatus 1 (Step 207) and to store the digital image data in the remote device 40 (Step 208) or send the digital image data to the remote device 40 (Step 209).

Although the present invention has been described with reference to the preferred embodiment thereof, it will be understood that the invention is not limited to the details thereof. Various substitutions and modifications have been suggested in the foregoing description, and other will occur to those of ordinary skill in the art. Therefore, all such substitutions and modifications are intended to be embraced within the scope of the invention as defined in the appended claims.

1. A method for remotely controlling a medical apparatus, said medical apparatus is used to take internal images of a human body and having control command sets that comply with the DICOM specifications and is capable of generating digital image data conforming to the DICOM specifications, said method comprising the steps of:
(a) building a server and connecting said server to said medical apparatus;
(b) building at least one remote device and connecting said remote device to said server;
(c) said remote device sending at least one DICOM control command to said medical apparatus via said server; and
(d) commanding said medical apparatus to take internal images of the human body for generating said digital image data.

2. The method for remotely controlling a medical apparatus as claimed in claim 1, wherein said medical apparatus is an angiography, an ultrasound unit, an endoscope, an intraoral camera or a CAT scanner.

3. The method for remotely controlling a medical apparatus as claimed in claim 1, wherein said server uses said control command that comply with the DICOM specifications to connect with said medical apparatus in said Step (a).

4. The method for remotely controlling a medical apparatus as claimed in claim 1, further comprising, after said Step (b), the following step:
said remote device executing a remote control program to generate at least one DICOM control command.

5. The method for remotely controlling a medical apparatus as claimed in claim 1, further comprising, after said Step (c), the following step:
said server executing a communication program to perform control communication with said medical apparatus so as to control said medical apparatus for performing capture, storage or transmission of said digital image data.

6. The method for remotely controlling a medical apparatus as claimed in claim 1 further comprising, after said Step (d), the following step:
storing the said digital image data into said server.

7. The method for remotely controlling a medical apparatus as claimed in claim 1, further comprising, after said Step (e), the following step:
sending said digital image data to said remote device.

8. A method for remotely controlling a medical apparatus, said medical apparatus is used to take images of the inside of human body for generating medical image signals, said method comprising the steps of:
(a) building a digital conversion device and connecting the said digital conversion device to said medical apparatus in order to convert said medical image signals into digital image data that comply with the DICOM specifications;
(b) building at least a remote device and connecting said remote device to said digital conversion device;
(c) said remote device sending at least a DICOM control command to said digital conversion device; and
(d) controlling said digital conversion device to performcapture, storage or transmission of said digital image data.

9. The method for remotely controlling a medical apparatus as claimed in claim 8, wherein said medical apparatus is an angiography, an ultrasound unit, an endoscope, an intraoral camera or a CAT scanner.

10. The method for remotely controlling a medical apparatus as claimed in claim 8, wherein said digital conversion device in said Step (a) has an image capturing unit and a storage unit therein, and said image capturing unit is used to capture said medical image signals, convert said medical image signals into digital image data that comply with the DICOM specifications, and store said digital image data into said storage unit.

11. The method for remotely controlling a medical apparatus as claimed in claim 8 further comprising, before said Step (b), the following steps:
building a server and connecting said server to said digital conversion device; and
connecting said remote device to said server and then to said digital conversion device via said server.

12. The method for remotely controlling a medical apparatus as claimed in claim 8 further comprising, after said Step (b), the following step:
said remote device executing a remote control program to generate at least one DICOM control command.

13. The method for remotely controlling a medical apparatus as claimed in claim 8 further comprising, after said Step (c), the following step:
said remote device executing a communication program to perform control communication with said digital conversion device so as to control said digital conversion device for performing capture, storage or transmission of said digital image data.

14. The method for remotely controlling a medical apparatus as claimed in claim 8 further comprising, after said Step (d), the following step:
storing said digital image data into said remote device; and
sending said digital image data to said remote device.

15. A device for remotely controlling a medical apparatus, said device comprising:
at least a medical apparatus capable of taking internal images of a human body for generating medical image signals;
a server connected to said medical apparatus and used to control said medical apparatus for generating digital image data conforming to DICOM specifications;
an image storage database connected to said server and used to store said digital image data; and
at least a remote device connected to said server and capable of generating at least a DICOM control command to control said medical apparatus via said server for performing capture, storage or transmission of said digital image data.

16. The device for remotely controlling a medical apparatus as claimed in claim 15, wherein said medical apparatus is an angiography, an ultrasound unit, an endoscope, an intraoral camera or a CAT scanner.

17. The device for remotely controlling a medical apparatus as claimed in claim 15 further comprising a digital conversion device connected to said medical apparatus and said server and used to convert said medical image signals into digital image data conforming to the DICOM specifications and then to send said digital image data to said server.

18. The device for remotely controlling a medical apparatus as claimed in claim 17, wherein said digital conversion device further comprises:
an image capturing unit connected to said medical apparatus and used to capture said medical image signals and convert said medical image signals into digital image data conforming to the DICOM specifications;
a storage unit connected to said image capturing unit and used to store said digital image data; and
a network interface unit connected to said image capturing unit and capable of connecting to said server via a network to send said digital image data to said server.

19. The device for remotely controlling a medical apparatus as claimed in claim 15, wherein said server comprises:
a program storage unit storing at least a communication program, said communication program being used to perform control communication with said medical apparatus or a digital conversion device for controlling capture, storage or transmission of said digital image data;
a processing unit connected to said program storage unit and used to execute said communication program to perform control communication with said medical apparatus; and
a network interface unit connected to said processing unit and capable of connecting to said medical apparatus and said remote device via a network for receiving or transmitting said digital image data.

20. The device for remotely controlling a medical apparatus as claimed in claim 15, wherein said remote device at least comprises:
a memory unit for storing at least a remote control program;
a processing unit connected to said memory unit and used to execute said remote control program for generating at least a DICOM control command;
a network interface unit connected to said processing unit and capable of connecting to said server via a network and transmitting said DICOM control command to said server or receiving said digital image data; and
display unit connected to said processing unit and used to display said digital image data.
